(19) United States
(12) Patent Application Publication
(10) Pub. No.: US 2021/0244805 A1
(43) Pub. Date: Aug. 12, 2021

(54) PHARMACEUTICAL FOR PREVENTION OF BLEOMYCIN-INDUCED PULMONARY FIBROSIS

(71) Applicant: UNIVERSITY OF SOUTH CAROLINA, COLUMBIA, SC (US)

(72) Inventors: MICHAEL WYATT, COLUMBIA, SC (US); CHLOE E. LEBEGUE, SIMPSONVILLE, SC (US)

(21) Appl. No.: 17/166,566

(22) Filed: Feb. 3, 2021

Related U.S. Application Data
(60) Provisional application No. 62/972,816, filed on Feb. 11, 2020.

(51) Int. Cl. A61K 38/48

(52) U.S. Cl. CPC A61K 38/4873; C12Y 304/2204

(57) ABSTRACT

Disclosed is an inhalable pharmaceutical composition that includes bleomycin hydrolase. The inhalable pharmaceutical composition can encompass a dry inhalable powder, aerosol, or a liquid-based composition for nebulization. Also disclosed are methods an for forming an inhalable pharmaceutical comprising bleomycin hydrolase. Disclosed compositions can be used in prevention of bleomycin-induced pulmonary fibrosis.

### CROSS REFERENCE TO RELATED APPLICATION

This application claims filing benefit of U.S. Provisional Patent Application Ser. No. 62/972,816, having a filing date Feb. 11, 2020, which is incorporated herein by reference in its entirety.

### BACKGROUND

Bleomycin is a chemotherapy agent used in the treatment of several different cancers, including Hodgkin's lymphoma, non-Hodgkin's lymphoma, testicular cancer, ovarian cancer, and cervical cancer, among others. Unfortunately, the FDA currently lists a black box warning for bleomycin as inducing a decrease in lung function, which usually presents secondary to the development of pulmonary fibrosis. Bleomycin-induced pulmonary fibrosis may occur in as many as 10% of patients treated with bleomycin and may be fatal. The potential for development of bleomycin-induced pulmonary fibrosis restricts use of the product, especially in children, and limits the use of bleomycin for long-term and maintenance therapy. The pathological mechanism of the development of pulmonary fibrosis is not fully understood; however, it is thought to be due to the lack of the human enzyme that deactivates bleomycin, bleomycin hydrolase (BLMH), in pulmonary tissue.

Bleomycin-induced pulmonary fibrosis is currently managed with corticosteroids and bronchodilators, with limited results. The art is lacking methods and compositions for use in preventing bleomycin-induced pulmonary fibrosis.

### SUMMARY

According to one embodiment, disclosed is an inhalable pharmaceutical composition that includes BLMH. For instance, an inhalable pharmaceutical composition can be in the form of a dry powder, an aerosol, or a liquid suitable for inhalation via a dry inhaler, a pressurized inhaler, or a nebulizer.

Also disclosed are methods for forming an inhalable pharmaceutical comprising BLMH. For instance, methods can include formation of a particulate including purified human BLMH, and combination of a carrier material with the particulate, either as a component of a particulate or as a separate material that can be in the form of a solid particulate or a fluid.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

Disclosed are pharmaceutical compositions and methods for preparation thereof that can prevent pulmonary side-effects of bleomycin therapies. More specifically, disclosed pharmaceutical compositions provide an inhalable form of the enzyme BLMH. Delivery of BLMH directly to pulmonary tissue by use of an inhalable pharmaceutical composition as disclosed can be utilized to deactivate bleomycin in the lungs and prevent undesirable side effects of bleomycin therapies, and particularly, bleomycin-induced pulmonary fibrosis.

BLMH is a cytoplasmic cysteine peptide hydrolase with a molecular weight of 250-280 kDa that is present in the cytoplasm as a homohexameric protein. In addition to its aminopeptidase activity, it has homocysteine-thiolactonase activity. The complete biological function of bleomycin hydrolase is not completely clear, but it is known to inactivate the glycopeptide anticancer agent bleomycin. It contains the characteristic active site residues of the cysteine proteolytic enzyme papain superfamily, and the coding gene is present at locus 17q11.2 in humans.

BLMH as may be utilized in disclosed compositions can include human BLMH proteins as have been described previously (see, e.g., NCBI reference sequences NP_000377.1, EAW51224, CAA63078, AAH03616.1, AAP35664.1, Q13867.1).

While BLMH of disclosed pharmaceutical compositions can include recombinantly produced human BLMH, the compositions are not limited to such, and it should be understood that BLMH of disclosed pharmaceutical compositions is not limited to any particular BLMH proteins. Other BLMH proteins, e.g., synthetic or animal-based proteins that can inactivate bleomycin are also encompassed herein. For instance, a pharmaceutical composition can include an extracted BLMH as may be extracted from human or host cell sources, as well as an analog BLMH, which may be synthetically or recombinantly produced. As utilized herein, the term “analog BLMH” generally refers to a non-natural BLMH substantially similar to a natural BLMH and having substantially the same or superior biological activity. The term “analog” is intended to include derivatives (e.g., chemical derivatives) of a biologically active polypeptide, which derivatives exhibit a qualitatively similar effect to that of an unmodified polypeptide.

In one embodiment, formation of disclosed pharmaceutical compositions can include production of BLMH. For instance, human BLMH can be initially produced according to known recombinant protein formation techniques. In such an embodiment, human cDNA for BLMH as known in the art can be inserted into a plasmid and the plasmid can then be propagated in a suitable production species, e.g., a bacterial production species such as E. coli. Upon expression, the BLMH can be isolated, collected, purified, and tested for efficacy, according to standard practice.

In one embodiment, a formation method need not include production of the BLMH, and a pharmaceutical composition can be formed to include a purified recombinant BLMH as is available in the market. By way of example, purified E. coli derived recombinant human BLMH is available from R&D Systems™, enQuire BioReagents, or MyBioSource, Inc., among others.

The pharmaceutical composition can include the BLMH in a formulation that is suitable for direct delivery to pulmonary tissue via inhalation, where it can deactivate bleomycin in the tissue. An inhalable pharmaceutical composition can be either a wet or dry formulation that can be administered via the pulmonary route into the lungs, generally by drawing the composition into the lungs through the mouth, though nasal administration is also contemplated herein.

In one embodiment, an inhalable composition can incorporate BLMH as a component of a particle or droplet in which the particle/droplet size facilitates penetration throughout the lungs. As utilized herein, the term “particle” generally refers to a porous or nonporous solid, while the term “droplet” generally refers to a single fluid component of a multi-component fluid with each droplet discontinuous from other droplets and can include, without limitation, liquid droplets in a gaseous or liquid continuous phase that is immiscible with the liquid of the droplets, gaseous bubbles in a liquid continuous phase, micelles in a liquid or gaseous continuous phase, etc. In one embodiment, a pharmaceutical composition designed to be inhaled from a dry powder inhaler can include dry particles comprising BLMH. In one embodiment, an inhalable composition can include particles or droplets comprising BLMH suspended in a propellant, e.g., in the form of an aerosol. In one embodiment, an inhalable composition can be a suspension of droplets or particles comprising BLMH held in a liquid carrier that can be intended for administration by use of a liquid nebulizer system. In such embodiments, a pharmaceutical composition can incorporate an aqueous liquid carrier, a nonaqueous liquid carrier, or can include a combination of an aqueous and nonaqueous carrier.

A pharmaceutical composition can include individual particles or droplets having a size that can permit penetration into the alveoli of the lungs, generally about 10 μm or less in size, about 7.5 μm or less in size, or about 5 μm or less in size in some embodiments. For instance, particles/droplets comprising BLMH can be from about 0.1 μm to about 5 μm in some embodiments. However, in some embodiments, larger structures are encompassed herein. For instance, when considering aerodynamically light particles (e.g., having a bulk density of about 0.5 g/cm³ or less) for delivery as a dry powder formulation, a pharmaceutical composition can carry larger particles; for instance, having a size of from about 5 μm to about 30 μm.

In illustrative embodiments, the majority and/or the mean size of the particles or droplets can range from equal to or greater than about 1, 2.5, 5, 10, 15 or 20 μm and/or equal to or less than about 25, 30, 40, 45, 50, 60, 75, or 100 μm (including all combinations of the foregoing). Representative examples of suitable ranges for the majority and/or mean particle/droplet size include, without limitation, from about 5 μm to about 100 μm, from about 1 μm to about 10 μm, or from about 0.1 μm to about 5 μm, which facilitate the deposition of an effective amount of BLMH in pulmonary tissue.

Particles incorporating BLMH can be prepared by any suitable formation technique, examples of which include, without limitation, lyophilization (freeze-drying), vacuum drying, or evaporative drying of a suitable BLMH-containing solution under conditions to produce the desired structure. To achieve a targeted particle size, the initially formed particles may be subjected to a size-reducing process such as micronization, grinding, or milling, e.g., jet milling. The desired size fraction may then be separated out by air classification or sieving.

By way of example, in one embodiment, a particulate in a desired size range can be formed by spray drying in which pure, as-received BLMH can be dissolved in a physiologically acceptable aqueous buffer, e.g., a citrate buffer having a pH in the range from about 2 to 9. The BLMH can generally be dissolved at a concentration from about 0.01 wt. % to about 1 wt. %; for instance, from about 0.1 wt. % to about 0.2 wt. %. The solution may then be spray dried in conventional spray drying equipment from commercial suppliers, resulting in a substantially amorphous particulate product.

The total content of solvent or solvents being employed in a solution being spray dried can generally be about 99 wt. % or greater and the content of other components (BLMH and other ingredients) present in the solution being spray dried can generally be about 1.0 wt. % or less; for instance, from about 0.05 wt. % to about 0.5 wt. % in some embodiments.

Suitable spray-drying techniques are generally known in the art. For instance, heat from a hot gas such as heated air, argon, or nitrogen can be used to evaporate the solvent from droplets formed by atomizing a continuous liquid feed. In one embodiment, a rotary atomizer can be employed.

In one embodiment, particles incorporating BLMH can be substantially free from any other biologically active components, pharmaceutical carriers, and the like. Such “neat” pharmaceutical compositions may include minor components, such as preservatives, present in small amounts, generally about 5 wt. % or less or about 2 wt. % or less, in some embodiments.

In one embodiment, particles incorporating BLMH can exhibit a sustained release profile. A sustained released profile can provide for prolonged residence of the BLMH in the pulmonary tissue and can thereby increase the amount of time during which therapeutic levels of the BLMH are present in the local environment. Consequently, patient compliance and comfort can be increased by not only reducing frequency of dosing, but by providing a therapy which is more amenable and efficacious to patients.

Sustained release profiles can be provided in one embodiment through inclusion in the particles of a phospholipid carrier material. For example, particles can include a phospholipid carrier material in an amount up to about 90 wt. %; for instance, from about 10 wt. % to about 60 wt. % in some embodiments. Examples of phospholipids can include, without limitation, phosphatidic acids, phosphatidylcholines, phosphatidylalkanolamines such as a phosphatidylethanolamines, phosphatidylglycerols, phosphatidylserines, phosphatidylinositols, and combinations thereof. To impart a sustained release profile, the phase transition temperature of a specific phospholipid can be below, around, or above the expected physiological body temperature of a subject. In one embodiment, the phase transition temperature of a phospholipid carrier material can be from about 30° C. to about 50° C. (e.g., within about ±10° C. of the normal body temperature of patient). By selecting a phospholipid or a combination of phospholipids according to their phase transition temperature, the particles can be tailored to have controlled release properties. For example, by administering particles which include a phospholipid or combination of phospholipids that have a phase transition temperature higher than the patient's body temperature, the release of BLMH may be slowed. On the other hand, rapid release can be obtained by including phospholipids having lower transition temperatures.

In one embodiment, a dry particulate pharmaceutical composition can be combined with pharmaceutical carrier materials or excipients which are suitable for respiratory and pulmonary administration. Such carrier materials may serve simply as bulking agents to control BLMH concentration in the pharmaceutical composition, or may provide one or more alternate or additional functions to a pharmaceutical composition. For instance, a carrier material can enhance the stability of the pharmaceutical composition and/or can improve the dispersibility of the pharmaceutical composition within a dry powder inhaler and/or can improve flowability and consistency of the powder, which can facilitate manufacturing and powder filling of an inhaler.

Dry carrier materials may be amorphous, crystalline, or a combination of amorphous and crystalline. Exemplary dry carrier materials include, without limitation, carbohydrates, including monosaccharides (e.g., fructose, galactose, glucose, D-mannose, sorbose, and the like); disaccharides (e.g., lactose, trehalose, cellobiose, and the like); cyclodextrins (e.g., 2-hydroxypropyl-β-cyclodextrin); polysaccharides (e.g., raffinose, maltodextrins, dextrans, starch, and the like); celluloses (e.g., methyl cellulose, carboxymethylcellulose, hydroxypropyl cellulose, and the like); xanthan gum; carbomer; alginate; polyvinyl alcohol; acacia; chitosans; amino acids (e.g., glycine, arginine, aspartic acid, glutamic acid, cysteine, lysine, and the like); organic salts prepared from organic acids and bases (e.g., sodium citrate, sodium ascorbate, magnesium gluconate, sodium gluconate, tromethamine hydrochloride, and the like); peptides and proteins (e.g., aspartame, human serum albumin, gelatin, and the like); alditols (e.g., mannitol, xylitol, and the like); or combination of two or more different dry carrier materials.

When included in a dry pharmaceutical composition, in one embodiment, a dry carrier material may be combined with the BLMH component prior to particle formation, e.g., by adding a carrier material to a buffer solution prepared for spray drying. In that way, the carrier material will be formed simultaneously with, and as a component of, the particles that also carry the BLMH. Typically, when individual particles include both a carrier material and BLMH, the particles can include the BLMH component in an amount from about 5 wt. % to about 95 wt. %, or from about 20 wt. % to about 80 wt. % in some embodiments. The particles can generally include the carrier material component in an amount of from about 5 wt. % to about 95 wt. %, or from about 20 wt. % to about 80 wt. % in some embodiments. However, particles can include additional components as known in the art, examples of which are discussed herein.

In one embodiment, a dry carrier material may additionally or alternatively be separately prepared in a dry powder form and combined with the particles that include BLMH by blending. In this embodiment, the particles that carry BLMH may also incorporate a carrier material that can be the same or different as the carrier material of the particles that do not include BLMH. Separately prepared carrier material particulates can be crystalline, and can avoid water absorption, but can in some cases be amorphous or mixtures of crystalline and amorphous phases. The size of separate carrier particles can be the same or differ from the size of the BLMH-containing particles. For instance, in some embodiments, carrier material particles can be sized to improve the flowability of the dry particulate. In one embodiment, carrier materials particles can have a size of from about 25 μm to about 100 μm. In one embodiment, the carrier material particles can be larger than the particles that include BLMH and, as such, may not penetrate into the alveolar region of the lung and can optionally be separated from the BLMH-containing particles, for instance prior to or during inhalation. For example, the larger carrier material particles can remain in an inhaler while the BLMH-containing particles can be delivered to pulmonary tissue.

In some embodiments, the pharmaceutical composition can be in the form of a suspension or aerosol in which the pharmaceutical composition can include droplets or particles that include BLMH in an aqueous or non-aqueous fluid carrier material. By way of example, an aqueous suspension can include droplets in the form of micelles of lipophilic substances, liposomes (phospholipid vesicles/membranes) and/or a fatty acid (e.g., palmitic acid) droplets that incorporate BLMH. In one embodiment, a pharmaceutical composition can be in the form of a solution or suspension in which the droplets or particles that carry BLMH are capable of dissolving in a fluid secreted by the pulmonary tissue to which it is administered, applied and/or delivered, which can advantageously enhance absorption of the BLMH. For example, a pharmaceutical composition or a BLMH-containing component thereof can be least partially, or even substantially (e.g., at least 80%, 90%, 95% or more) soluble in a fluid that is secreted by pulmonary tissue so as to facilitate BLMH absorption by the tissue. In some embodiments, a pharmaceutical composition can include one or more additives that foster dissolution of a BLMH-containing component within secretions, such as, and without limitation to, fatty acids (e.g., palmitic acid), gangliosides (e.g., GM-1), phospholipids (e.g., phosphatidylserine), and emulsifiers (e.g., polysorbate 80). For instance, in one embodiment, BLMH-containing droplets or particles can be configured to dissolve in a fluid secreted by pulmonary tissue or degrade over time following delivery to pulmonary tissue and thereby release BLMH at the tissue, where it can be e.g., absorbed into pulmonary capillaries.

An aqueous pharmaceutical composition can incorporate an aqueous carrier material and can include, but is not limited to, aqueous gels, aqueous suspensions, aqueous microsphere suspensions, aqueous microsphere dispersions, aqueous liposomal dispersions, aqueous micelles of liposomes, aqueous microemulsions, and any combination of the foregoing, or any other aqueous composition that can carry BLMH droplets or particles that can release BLMH to pulmonary tissue upon delivery.

A nonaqueous pharmaceutical composition can incorporate a nonaqueous (i.e., organic) carrier material and can include, but is not limited to, nonaqueous gels, nonaqueous suspensions, nonaqueous microsphere suspensions, nonaqueous microsphere dispersions, nonaqueous liposomal dispersions, nonaqueous emulsions, nonaqueous microemulsions, and any combination of the foregoing, or any other nonaqueous composition that can carry BLMH droplets or particles that can release BLMH to pulmonary tissue upon delivery.

An aqueous or non-aqueous fluid carrier material can include, by way of example and without limitation, water, ethanol, polyol (for example, glycerol, propylene glycol and liquid polyethylene glycol, and the like), suitable mixtures thereof, and/or vegetable oils. A desired fluidity of a fluid-based pharmaceutical suspension can be maintained by the use of an additive such as, for example, a surfactant or lecithin, which can also assist in maintaining particle size in a dispersion.

In one embodiment, a pharmaceutical composition can include particles incorporating BLMH in the form of an aerosol. An aerosol pharmaceutical composition can be in the form of a suspension or solution and typically includes a suitable propellant, e.g., a low boiling point, highly volatile propellant. In one embodiment, an aerosol can include a suspension including a BLMH-containing particulate in a propellant. In another embodiment, an aerosol can include BLMH dissolved or carried in a carrier material that is within the propellant. In one embodiment, an aerosol pharmaceutical composition can be provided within a valved container and maintained under pressure. In some embodiments, a container can incorporate a metering valve and can be provided with a removable or permanent mouthpiece.

When low boiling point propellants are used, the propellants can be maintained within a pressurized container in a liquid state. Upon actuation of the valve, the propellant will expand and volatilize, thus forcing the BLMH component from the container along with the propellant. Moreover, a low boiling point propellant can flash and evaporate as the materials exit the container, thus completely, or essentially completely, removing the propellant from the aerosol and thereby avoiding delivery of the propellant to the pulmonary tissue of the subject.

Examples of low boiling point propellants as may be incorporated in an aerosol pharmaceutical composition can include, without limitation, hydrofluoroalkanes such as chlorofluorocarbons, e.g., trichlorofluoromethane; dichlorodifluoromethane; trichlorofluoromethane; dichlorotetrafluoroethane; 1,1,1,2-tetrafluoroethane; 1,1,1,2,3,3,3-heptafluoro-n-propane; or any combination thereof.

An aerosol pharmaceutical composition can include additional excipients typically associated with such compositions, such as, for example, and without limitation, surfactants, such as oleic acid or lecithin, and co-solvents, such as ethanol.

In one embodiment, a pharmaceutical composition can be in the form of a solution or suspension configured for inhalation, for instance by nebulization. For instance, a pharmaceutical composition can include an aqueous carrier material in conjunction with BLMH-containing particles, droplets, or BLMH dissolved in the carrier material with the addition of agents such as acid or alkali, buffer salts, isotonic adjusting agents or antimicrobial agents.

A pharmaceutical composition configured for inhalation by nebulization can be sterilized by filtration or heating in an autoclave, or presented as a non-sterile product. A nebulizer can be utilized to supply the pharmaceutical as a fine spray, mist, or suspension created from the as-prepared formulation.

A pharmaceutical composition can include one or more additional components that can provide desirable characteristics to the composition. By way of example, a pharmaceutical composition can include one or more solvents, dispersion media, coatings, antibacterials, antivirals and/or antifungals, isotonic and absorption delaying agents and the like. The use of such additives for pharmaceutically active substances is well known in the art. Except insofar as any conventional medium or agent is incompatible with the active ingredient, use thereof in the pharmaceutical compositions is encompassed herein.

Additional additives as may be incorporated in a pharmaceutical composition can include, but are not limited to, carriers, excipients, viscosity-increasing agents, preservers, stabilizers, anti-oxidants, binders, disintegrants, humectants, lubricants, colorants, flavoring agents, corrigents, suspend molding agents, emulsifying agents, solubilizers, buffering agents, tonicity agents, detergents, soothing agents, sulfur-containing reducing agents, etc.

In embodiments of the invention, the pH of a pharmaceutical composition can be designed so that the internal environment of the pulmonary tissue to which the composition is delivered is on the acidic-to-neutral side following administration. Such an embodiment can provide the BLMH of the composition in an un-ionized form for absorption, can prevent growth of pathogenic bacteria (which is more likely to occur in an alkaline environment), and can reduce the likelihood of irritation of the pulmonary tissue.

In one embodiment, a pharmaceutical composition can be isotonic to slightly hypertonic, e.g., having an osmolarity ranging from about 150 to 550 mOsM. In other embodiments, the pharmaceutical composition is isotonic, having, e.g., an osmolarity ranging from approximately 150 to 350 mOsM.

A pharmaceutical composition can optionally include an absorption enhancer, such as an agent that inhibits enzyme activity, reduces mucous viscosity or elasticity, decreases mucociliary clearance effects, opens tight junctions, and/or solubilizes the active compound. Chemical enhancers are known in the art and include chelating agents (e.g., EDTA), fatty acids, bile acid salts, surfactants, and/or preservatives. Enhancers for penetration can be particularly useful when formulating compounds that exhibit poor membrane permeability, lack of lipophilicity, and/or are degraded by aminopeptidases. The preferred concentration of an absorption enhancer in a pharmaceutical composition will vary as is known depending upon the agent selected and the complete formulation of the pharmaceutical composition.

To extend shelf life, preservatives can be included in a pharmaceutical composition. Exemplary preservatives include, but are not limited to, benzyl alcohol, parabens, thimerosal, chlorobutanol and benzalkonium chloride, and combinations of the foregoing. As is known, the concentration of a preservative in a pharmaceutical composition can vary depending upon the preservative used, the formulation, and the like. In representative embodiments, a preservative can be present in an amount of about 2 wt. % or less.

Disclosed pharmaceutical compositions can beneficially prevent bleomycin-induced pulmonary fibrosis and can allow for an expansion of the role of bleomycin in clinical practice. As previously discussed, bleomycin is an effective chemotherapeutic agent, but is limited in practice due to concern for bleomycin-induced pulmonary fibrosis, which can be fatal, as well as prevention of other pulmonary toxicities. As such, in one embodiment, disclosed pharmaceutical compositions can be administered in conjunction with bleomycin therapeutics. When co-administered, bleomycin can be provided in standard therapeutic amounts and administration routes, including, and without limitation to, intravenously, by injection into a muscle or under the skin. Such co-administration can allow patients to continue with bleomycin-based therapies, e.g., bleomycin-based cancer chemotherapy regiments, unhindered.

While certain embodiments of the disclosed subject matter have been described using specific terms, such description is for illustrative purposes only, and it is to be understood that changes and variations may be made without departing from the spirit or scope of the subject matter.

What is claimed is:

1. A pharmaceutical composition comprising bleomycin hydrolase, wherein the pharmaceutical composition is an inhalable pharmaceutical composition.

2. The pharmaceutical composition of claim 1, the composition comprising particles or droplets that include the bleomycin hydrolase.

3. The pharmaceutical composition of claim 2, the particles or droplets further comprising a carrier material.

4. The pharmaceutical composition of claim 2, the pharmaceutical composition further comprising a carrier material.

5. The pharmaceutical composition of claim 4, wherein the carrier material comprises a particulate.

6. The pharmaceutical composition of claim 4, wherein the carrier material comprises a fluid.

7. The pharmaceutical composition of claim 6, wherein the carrier material comprises a liquid.

8. The pharmaceutical composition of claim 2, wherein the particles or droplets are about 10 micrometers or less in size.

9. The pharmaceutical composition of claim 2, the composition comprising particles that include the bleomycin hydrolase, wherein the particles are sustained release particles.

10. The pharmaceutical composition of claim 1, further comprising a propellant.

11. The pharmaceutical composition of claim 1, wherein the bleomycin hydrolase comprises human recombinant bleomycin hydrolase.

12. A method for forming a pharmaceutical composition, the method comprising incorporating a bleomycin hydrolase in a particulate or droplets, wherein the particulates or droplets are inhalable.

13. The method of claim 12, further comprising combining the bleomycin with a carrier material.

14. The method of claim 13, wherein the step of forming the particulate or droplets comprises the combining of the bleomycin with the carrier material.

15. The method of claim 13, wherein the step of combining the bleomycin with the carrier material comprises blending the particulate that comprises bleomycin with another particulate that comprises the carrier material.

16. The method of claim 13, wherein the carrier material comprises a fluid, the method including forming a suspension or a dispersion including the particulate or droplets comprising the bleomycin hydrolase carried in the carrier material.

17. The method of claim 12, the method comprising incorporating the bleomycin hydrolase in the particulate according to a spray drying process.

18. The method of claim 12, further comprising producing the bleomycin hydrolase by use of a bacterial expression system.

19. The method of claim 12, wherein the bleomycin hydrolase comprises human bleomycin hydrolase.

20. The method of claim 12, wherein the bleomycin hydrolase comprises recombinant bleomycin hydrolase.
